(19) United States
(12) Patent Application Publication
(10) Pub. No.: US 2009/0054782 A1
(43) Pub. Date: Feb. 26, 2009

(54) ULTRASOUND DIAGNOSTIC APPARATUS

(76) Inventor: Shinichi Amemiya, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/196,932

(22) Filed: Aug. 22, 2008

(30) Foreign Application Priority Data
Aug. 24, 2007 (JP) 2007-218372

(51) Int. Cl. A61B 8/14

(52) U.S. Cl. 600/459

(57) ABSTRACT

An ultrasound diagnostic apparatus includes: an ultrasound probe; a transceiver device which drives said ultrasound probe to scan a subject with an ultrasound beam; an image generating device which generates an ultrasound image on the basis of received signals obtained by said scanning; a display device which displays said ultrasound image; a first operation device and a second operation device to enable an operator to input instructions; and a control device which accepts instructions inputted from said first operation device and instructions inputted from said second operation device and controls said transceiver device, said image generating device and said display device in accordance with the instructions.

### CROSS REFERENCE TO RELATED APPLICATIONS

This application claims the benefit of Japanese Patent Application No. 2007-218372 filed Aug. 24, 2007, incorporated by reference herein in its entirety.

### BACKGROUND OF THE INVENTION

The subject matter disclosed herein relates to an ultrasound diagnostic apparatus, and more particularly to an ultrasound diagnostic apparatus that allows a plurality of operators to operate a single ultrasound diagnostic apparatus.

Ultrasound diagnostic apparatus each of which allows the body unit of the ultrasound diagnostic apparatus to be operated from an operation unit by radio communication are known (see for instance, Japanese Unexamined Patent Publication No. 2002-085405 and Japanese Unexamined Patent Publication No. 2003-153903).

For instance when a medical doctor is to train a sonographer (person whose task is to scan a subject with an ultrasound wave) on the job, it is preferable for the sonographer positioned near the patient and the doctor positioned somewhat away to be able to operate the same ultrasound diagnostic apparatus substantially at the same time.

However, as the operation unit and the body unit of the ultrasound diagnostic apparatus are in a one-to-one relationship in the conventional ultrasound diagnostic apparatus, there is a problem that the above-stated requirement cannot be met.

### BRIEF DESCRIPTION OF THE INVENTION

It is desirable that the problem described previously is solved.

The invention in its first aspect provides an ultrasound diagnostic apparatus including an ultrasound probe, a transceiver device which drives the ultrasound probe to scan a subject with an ultrasound beam, an image generating device which generates an ultrasound image on the basis of received signals obtained by the scanning, a display device which displays the ultrasound image, a first operation device and a second operation device to enable an operator to input instructions, and a control device which accepts instructions inputted from the first operation device and instructions inputted from the second operation device and controls the transceiver device, the image generating device and the display device in accordance with the instructions.

The ultrasound diagnostic apparatus according to the first aspect enables a first operator to operate the first operation device and a second operator to operate the second operation device and thereby to make a single ultrasound diagnostic apparatus operable by a plurality of operators. Therefore, for instance, a sonographer positioned near the patient and a doctor positioned somewhat away are enabled to operate the ultrasound diagnostic apparatus substantially at the same time.

Incidentally, an i-th (i≧3) operation device may be provided as well.

The invention in its second aspect provides the ultrasound diagnostic apparatus according to the first aspect wherein the types of instructions that can be inputted from the second operation device are the same as the types of instructions that can be inputted from the first operation device.

The ultrasound diagnostic apparatus according to the second aspect, as the types of instructions that can be inputted from the second operation device are the same as the types of instructions that can be inputted from the first operation device, is suitable for operation, for example, of a single ultrasound diagnostic apparatus by a plurality of operators of the same occupational function (e.g. two doctors).

The invention in its third aspect provides the ultrasound diagnostic apparatus according to the first aspect wherein only some of the types of instructions that can be inputted from the first operation device are permitted as the types of instructions that can be inputted from the second operation device.

The ultrasound diagnostic apparatus according to the third aspect, as the types of instructions that can be inputted from the first operation device and the types of instructions that can be inputted from the second operation device can be differentiated from each other, is suitable for operation, for example, of a single ultrasound diagnostic apparatus by a plurality of operators of different occupational functions (e.g. a sonographer and a doctor).

The invention in its fourth aspect provides the ultrasound diagnostic apparatus according to the first aspect wherein the types of instructions that can be inputted from the second operation device are switchable between the same as and some of the types of instructions that can be inputted from the first operation device.

The ultrasound diagnostic apparatus according to the fourth aspect, as the types of instructions that can be inputted from the first operation device are switchable between the same as and some of the types of instructions that can be inputted from the second operation device, is suitable for operation, for example, of a single ultrasound diagnostic apparatus both by a plurality of operators of the same occupational function (e.g. two doctors) and by a plurality of operators of different occupational functions (e.g. a sonographer and a doctor).

The invention in its fifth aspect provides the ultrasound diagnostic apparatus according to any of the first through fourth aspects wherein the types of instructions that can be inputted from the second operation device are selectable.

The ultrasound diagnostic apparatus according to the fifth aspect allows presetting of the types of instructions that can be inputted from the second operation device by selecting the types of instructions that can be inputted from the second operation device.

The invention in its sixth aspect provides an ultrasound diagnostic apparatus including an ultrasound probe, a transceiver device which drives the ultrasound probe to scan a subject with an ultrasound beam, an image generating device which generates an ultrasound image on the basis of received signals obtained by the scanning, a first display device and a second display device which display the ultrasound image, a first operation device and a second operation device to enable an operator to input instructions, and a control device which accepts instructions inputted from the first operation device and instructions inputted from the second operation device and controls the transceiver device, the image generating device, the first display device and the second display device in accordance with the instructions.

The ultrasound diagnostic apparatus according to the sixth aspect enables a plurality of operators to operate a single ultrasound diagnostic apparatus by having a first operator operate the first operation device while watching the first display device and having a second operator operate the second operation device while watching the second display device. Therefore, for instance, a sonographer positioned near the patient and a doctor positioned somewhat away can operate the same ultrasound diagnostic apparatus substantially at the same time.

Incidentally, an i-th (i≧3) display device and an i-th (i≧3) operation device may be provided as well.

The invention in its seventh aspect provides a ultrasound diagnostic apparatus according to the sixth aspect wherein inputting from the second operation device to the first display device is impossible.

The ultrasound diagnostic apparatus according to the seventh aspect, as the functions of the first display device cannot be altered from the second operation device, can assign the first display device for exclusive use by the first operation device.

The invention in its eighth aspect provides the ultrasound diagnostic apparatus according to the sixth or seventh aspect wherein inputting from the first operation device to the second display device is impossible.

The ultrasound diagnostic apparatus according to the eighth aspect, as the functions of the second display device cannot be altered from the first operation device, can assign the second display device for exclusive use by the second operation device.

The invention in its ninth aspect provides the ultrasound diagnostic apparatus according to any of the sixth through eighth aspects wherein the types of instructions that can be inputted from the second operation device do not include some of the types of instructions that can be inputted from the first operation device.

The ultrasound diagnostic apparatus according to the ninth aspect, as the types of instructions that can be inputted from the first operation device are differentiated from the types of instructions that can be inputted from the second operation device, is suitable for operation, for example, of a single ultrasound diagnostic apparatus by a plurality of operators different occupational functions (e.g. a sonographer and a doctor).

The invention in its tenth aspect provides the ultrasound diagnostic apparatus according to the ninth aspect wherein the types of instructions that can be inputted from the second operation device are selectable.

The ultrasound diagnostic apparatus according to the tenth aspect allows presetting of the types of instructions that can be inputted from the second operation device by selecting the types of instructions that can be inputted from the second operation device.

The invention in its eleventh aspect provides the ultrasound diagnostic apparatus according to the tenth aspect wherein some of the types of instructions that can be inputted from the first operation device cannot be selected as the types of instructions that can be inputted from the second operation device.

The ultrasound diagnostic apparatus according to the eleventh aspect, as the types of instructions that can be inputted from the first operation device can be differentiated from the types of instructions that can be inputted from the second operation device, is suitable for operation, for example, of a single ultrasound diagnostic apparatus by a plurality of operators different occupational functions (e.g. a sonographer and a doctor).

The invention in its twelfth aspect provides the ultrasound diagnostic apparatus according to the sixth aspect wherein the types of instructions that can be inputted from the second operation device include all of the types of instructions that can be inputted from the first operation device.

The ultrasound diagnostic apparatus according to the twelfth aspect, as the types of instructions that can be inputted from the first operation device are the same as the types of instructions that can be inputted from the second operation device, is suitable for operation, for example, of a single ultrasound diagnostic apparatus by a plurality of operators of the same occupational function (e.g. two doctors).

The invention in its thirteenth aspect provides the ultrasound diagnostic apparatus according to the sixth aspect wherein the types of instructions that can be inputted from the second operation device are switchable between all and not all of the types of instructions that can be inputted from the first operation device.

The ultrasound diagnostic apparatus according to the thirteenth aspect, as the types of instructions that can be inputted from the first operation device are switchable between the same as and different ones from the types of instructions that can be inputted from the second operation device, is adaptable for operation, for example, of a single ultrasound diagnostic apparatus both by a plurality of operators of the same occupational function (e.g. two doctors) and by a plurality of operators of different occupational functions (e.g. a sonographer and a doctor).

The invention in its fourteenth aspect provides the ultrasound diagnostic apparatus according to the thirteenth aspect wherein the types of instructions that can be inputted from the second operation device are selectable.

The ultrasound diagnostic apparatus according to the fourteenth aspect allows presetting of the types of instructions that can be inputted from the second operation device by selecting the types of instructions that can be inputted from the second operation device.

The invention in its fifteenth aspect provides the ultrasound diagnostic apparatus according to the fourteenth aspect wherein the types of instructions that can be inputted from the second operation device are selectable out of all of the types of instructions that can be inputted from the first operation device.

The ultrasound diagnostic apparatus according to the fifteenth aspect, as the types of instructions that can be inputted from the first operation device are switchable between the same as and different ones from the types of instructions that can be inputted from the second operation device, is adaptable for operation, for example, of a single ultrasound diagnostic apparatus both by a plurality of operators of the same occupational function (e.g. two doctors) and by a plurality of operators of different occupational functions (e.g. a sonographer and a doctor).

The invention in its sixteenth aspect provides the ultrasound diagnostic apparatus according to the fourteenth aspect wherein, when the types of instructions that can be inputted from the second operation device are to be selected in a state in which the types of instructions that can be inputted from the second operation device have been so switched as not to include some of the types of instructions that can be inputted from the first operation device, some of the types of instructions that can be inputted from the first operation device cannot be selected.

The ultrasound diagnostic apparatus according to the sixteenth aspect, as the types of instructions that can be inputted from the first operation device can be differentiated from the types of instructions that can be inputted from the second operation device, is suitable for operation, for example, of a single ultrasound diagnostic apparatus by a plurality of operators different occupational functions (e.g. a sonographer and a doctor).

The ultrasound diagnostic apparatus according to the invention enables a plurality of operators to operate a single ultrasound diagnostic apparatus.

The ultrasound diagnostic apparatus according to the invention can be utilized in a case where doctor is to train a sonographer on the job or where a plurality of doctors make an ultrasound diagnosis of a single patient.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of the ultrasound diagnostic apparatus pertaining to Embodiment 1.

FIG. 2 is a layout diagram showing the arrangement of the ultrasound diagnostic apparatus pertaining to Embodiment 1.

FIG. 3 shows a diagram of an example of setting screen of the ultrasound diagnostic apparatus pertaining to Embodiment 1.

FIG. 4 shows a diagram of an example of setting screen of the ultrasound diagnostic apparatus pertaining to Embodiment 2.

FIG. 5 is a block diagram showing the configuration of the ultrasound diagnostic apparatus pertaining to Embodiment 3.

FIG. 6 is a layout diagram showing the arrangement of the ultrasound diagnostic apparatus pertaining to Embodiment 3.

FIG. 7 shows a diagram of an example of setting screen of the ultrasound diagnostic apparatus pertaining to Embodiment 3.

FIG. 8 shows a diagram of an example of setting screen of the ultrasound diagnostic apparatus pertaining to Embodiment 4.

### DETAILED DESCRIPTION OF THE INVENTION

The invention will be described in further detail below with reference to modes for implementation thereof illustrated in the drawings. The invention should not be limited to the following modes.

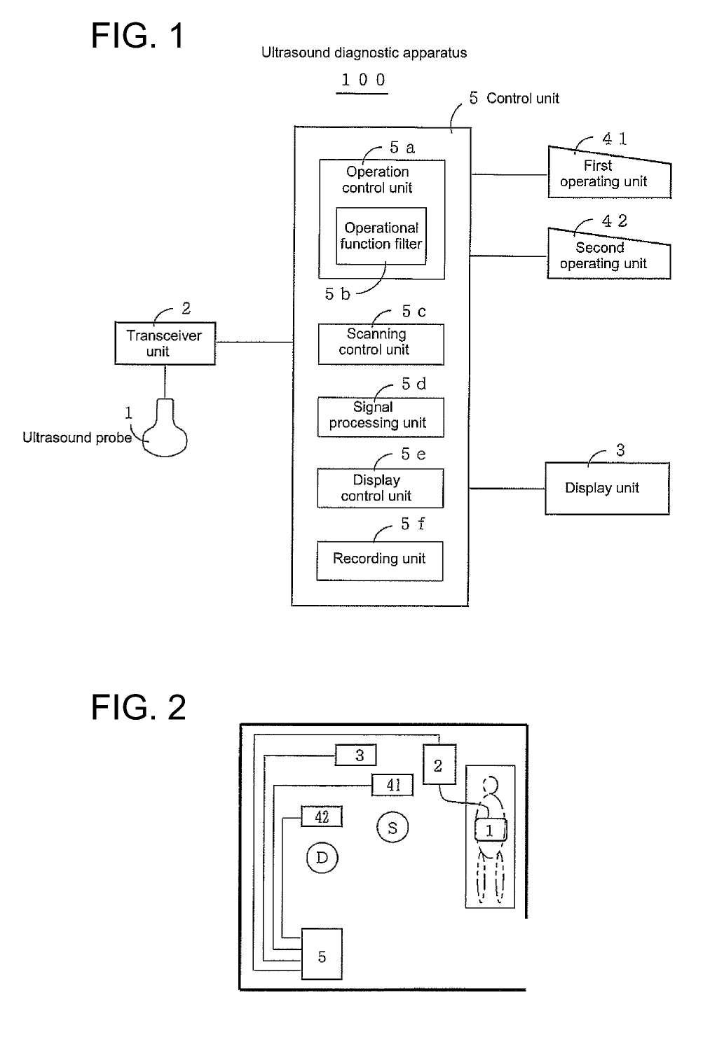

FIG. 1 is a diagram illustrating the configuration of an ultrasound diagnostic apparatus 100.

This ultrasound diagnostic apparatus 100 is provided with an ultrasound probe 1, a transceiver unit 2 which drives the ultrasound probe 1 to scan the inside of a subject with an ultrasound beam, a display unit 3 which displays ultrasound images and the like, a first operating unit 41 and a second operating unit 42 which enable the operator to input instructions and data, and a control unit 5 which exercises overall control.

The control unit 5 is provided with an operation control unit 5a for controlling operation-related functions including acceptance of inputs from the first operating unit 41 and the second operating unit 42, a scanning control unit 5c for controlling scanning-related functions including alteration of the mode (e.g. into B mode, CFM and so on) and alteration of scanning parameters, a signal processing unit 5d which performs processing of echo signals obtained by the ultrasound probe 1, generation of ultrasound images and the like, a display control unit 5e for controlling display-related functions including displaying of ultrasound images and messages on the display unit 3, and a recording unit 5f for recording ultrasound images and the like.

The operation control unit 5a includes an operational function filter 5b that limits the types of instructions that can be inputted from the second operating unit 42. Thus, while the first operating unit 41 can input all types of instructions to the ultrasound diagnostic apparatus 100, the second operating unit 42 can input only the types of instructions other than the instructions forbidden by the operational function filter 5b.

As shown in FIG. 2, the ultrasound probe 1, the transceiver unit 2 and the first operating unit 41 are arranged to match the position of a sonographer S near a bed (i.e. a patient). The second operating unit 42 is arranged to match the position of a doctor D somewhat away. The display unit 3 is so arranged to be visible by both the sonographer S and the doctor D.

The sonographer S scans the subject by using the ultrasound probe 1, the transceiver unit 2 and the first operating unit 41. Data obtained by scanning are sent from the transceiver unit 2 to the control unit 5. The control unit 5 generates an ultrasound image or the like on the basis of the data that have been sent, and sends it to the display unit 3.

The doctor D diagnoses the subject by using the display unit 3 and the second operating unit 42.

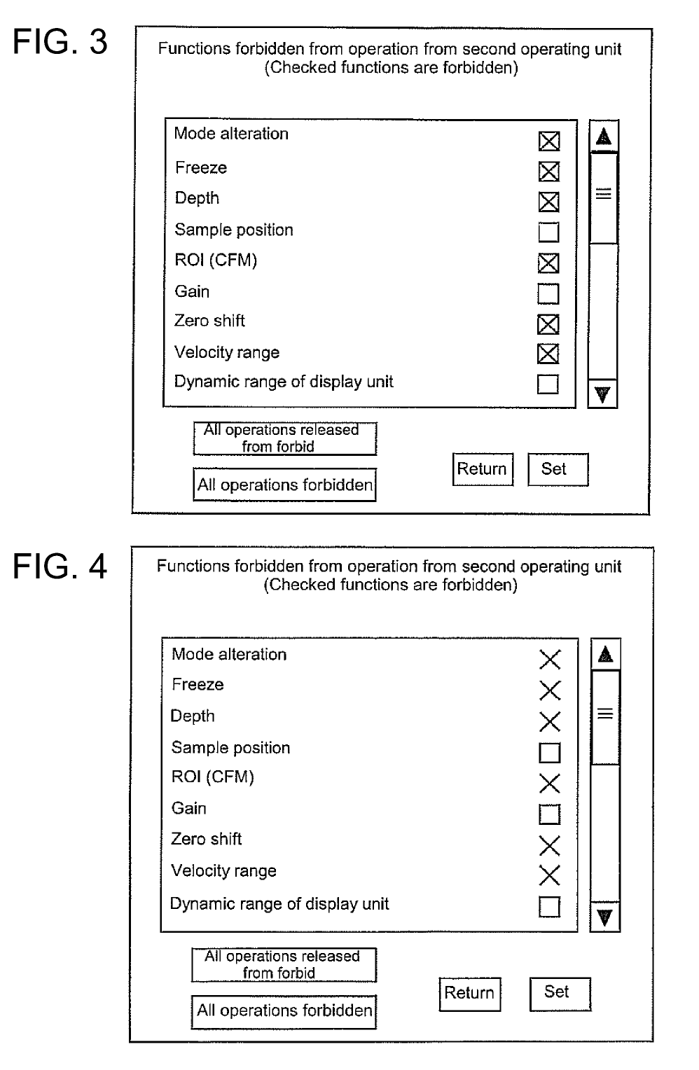

FIG. 3 shows a screen on which the types of instructions forbidden by the operational function filter 5b are preset.

Since instructions for the checked functions are forbidden, they cannot be inputted from the second operating unit 42. On the other hand, instructions not checked can be inputted from the second operating unit 42.

When an “All operations released from forbid” button is pressed, all the checks are undone, and therefore the first operating unit 41 and the second operating unit 42 become the same, and all the instructions for the ultrasound diagnostic apparatus 100 can be inputted from the second operating unit 42 as well.

When an “All operations forbidden” button is pressed, all the functions are checked, and therefore the second operating unit 42 is made ineffective.

The ultrasound diagnostic apparatus 100 of Embodiment 1 enables the ultrasound diagnostic apparatus 100 to be worked by using the plurality of operating units 41 and 42. Since it also enables the types of instructions that can be inputted from the first operating unit 41 and the types of instructions that can be inputted from the second operating unit 42 to be made either the same or different, the ultrasound diagnostic apparatus 100 can be adapted to operation both by a plurality of operators of the same occupational function (e.g. two doctors) and by a plurality of operators of different occupational functions (e.g. the sonographer S and the doctor D).

In an alternative embodiment, if checks for some of the functions are made irremovable out of all the instructions directed to the ultrasound diagnostic apparatus 100 as shown in FIG. 4, the types of instructions that can be inputted from the first operating unit 41 and the types of instructions that can be inputted from the second operating unit 42 can be differentiated.

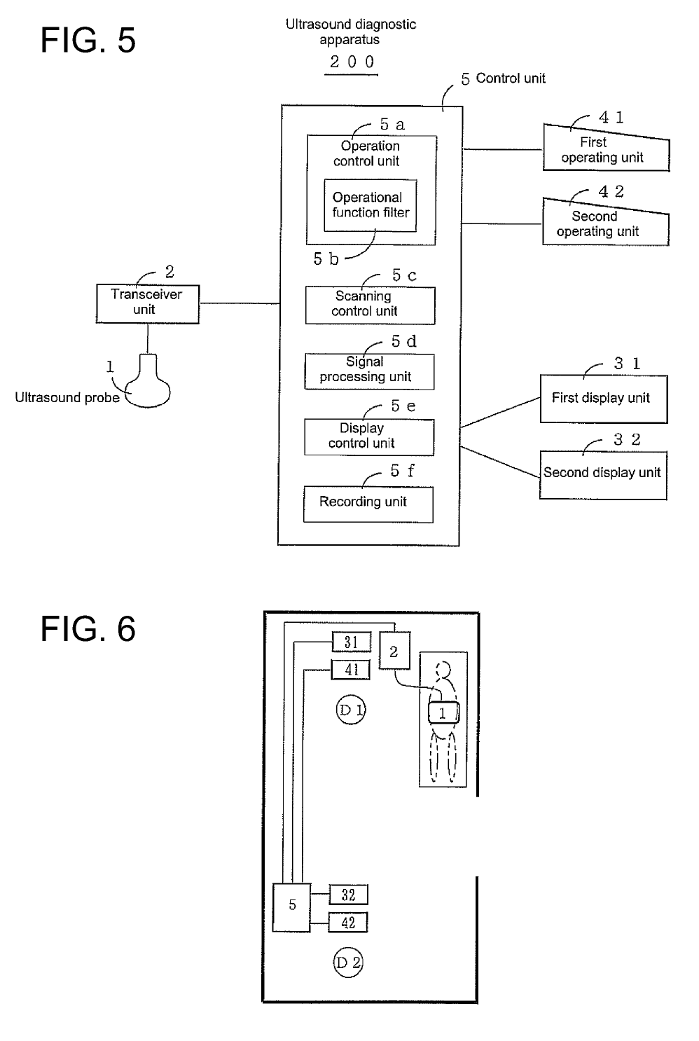

FIG. 5 illustrates the configuration of a second exemplary ultrasound diagnostic apparatus 200.

This ultrasound diagnostic apparatus 200 is provided with an ultrasound probe 1, a transceiver unit 2 which drives the ultrasound probe 1 to scan the inside of a subject with an ultrasound beam, a first display unit 31 and a second display unit 32 which display ultrasound images and the like, a first operating unit 41 and a second operating unit 42 which enable the operator to input instructions and data, and a control unit 5 which exercises overall control.

The control unit 5 is provided with an operation control unit 5a for controlling operation-related functions including acceptance of inputs from the first operating unit 41 and the second operating unit 42, a scanning control unit 5c for controlling scanning-related functions including alteration of the mode (e.g. into B mode, CFM and so on) and alteration of scanning parameters, a signal processing unit 5d which performs processing of echo signals obtained by the ultrasound probe 1, generation of ultrasound images and the like, a display control unit 5e for controlling display-related functions including displaying of ultrasound images and messages on the first display unit 31 and the second display unit 32, and a recording unit 5f for recording ultrasound images and the like.

The operation control unit 5a includes an operational function filter 5b that limits the types of instructions that can be inputted from the second operating unit 42. Thus, while the first operating unit 41 can input all types of instructions to the ultrasound diagnostic apparatus 100, the second operating unit 42 can input only the types of instructions other than the instructions forbidden by the operational function filter 5b.

As shown in FIG. 6, the ultrasound probe 1, the transceiver unit 2, the first display unit 31 and the first operating unit 41 are arranged to match the position of a doctor D1 near a bed (i.e. a patient). The second display unit 32 and the second operating unit 42 are arranged to match the position of a doctor D2 somewhat away.

The doctor D1 scans the subject by using the ultrasound probe 1, the transceiver unit 2 and the first operating unit 41. Data obtained by scanning are sent from the transceiver unit 2 to the control unit 5. The control unit 5 generates an ultrasound image or the like on the basis of the data that have been sent, and sends it to the first display unit 31 and the second display unit 32. The ultrasound image or the like that has been sent is displayed on the first display unit 31 and the second display unit 32.

The doctor D1 diagnoses the subject by using the first display unit 31 and the first operating unit 41.

The doctor D2 diagnoses the subject by using the second display unit 32 and the second operating unit 42.

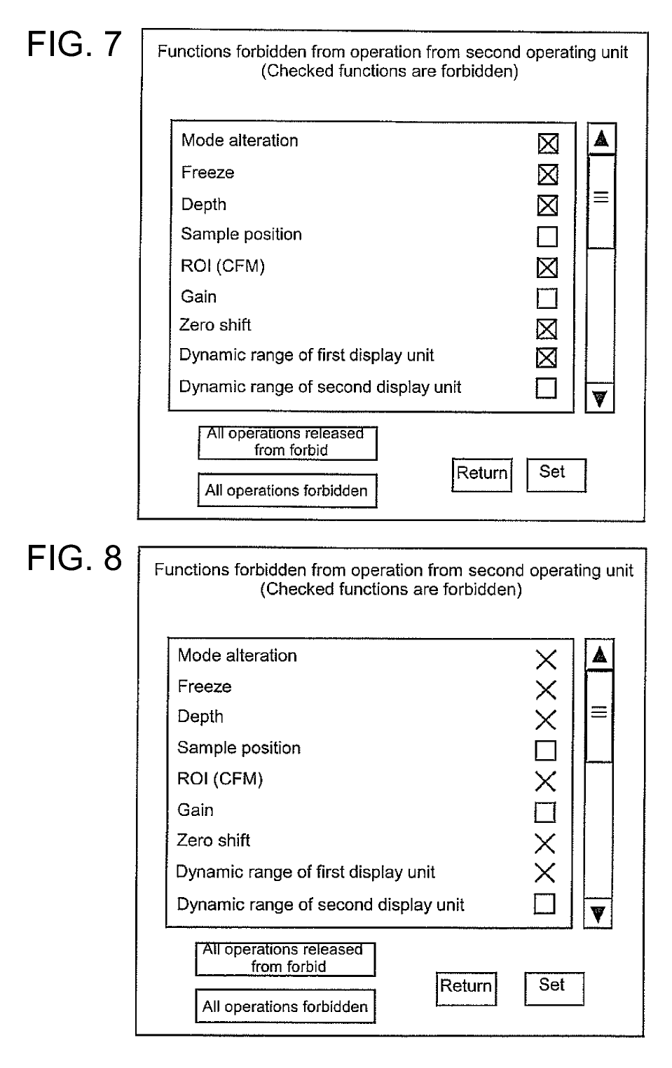

FIG. 7 shows a screen on the types of instruction forbidden by the operational function filter 5b are preset.

Since instructions for the checked functions are forbidden, they cannot be inputted from the second operating unit 42. On the other hand, instructions not checked can be inputted from the second operating unit 42.

When an “All operations released from forbid” button is pressed, all the checks are undone, therefore the first operating unit 41 and the second operating unit 42 become the same, and all the instructions for the ultrasound diagnostic apparatus 200 can be inputted from the second operating unit 42 as well.

When an “All operations forbidden” button is pressed, all the functions are checked, and therefore the second operating unit 42 is made ineffective.

The ultrasound diagnostic apparatus 200 of Embodiment 3 enables the ultrasound diagnostic apparatus 200 to be worked by using the plurality of display units 31 and 32 and the plurality of operating units 41 and 42. Since it also enables the types of instructions that can be inputted from the first operating unit 41 and the types of instructions that can be inputted from the second operating unit 42 to be made either the same or different, the ultrasound diagnostic apparatus 200 can be adapted to operation both by a plurality of operators of the same occupational function (e.g. two doctors D1 and D2) and by a plurality of operators of different occupational functions (e.g. a sonographer and a doctor).

In an alternative embodiment, if for some of the functions are made irremovable out of all the instructions directed to the ultrasound diagnostic apparatus 200 as shown in FIG. 8, the types of instructions that can be inputted from the first operating unit 41 and the types of instructions that can be inputted from the second operating unit 42 can be differentiated.

In one or more of the embodiments described above, it is possible to forbid instructions from the first operating unit 41 to the second display unit 32 and instructions from the second operating unit 42 to the first display unit 31.

It is also possible to assign the first display unit 31 for exclusive use by the first operating unit 41 and the second display unit 32 for exclusive use by the second operating unit 42.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus comprising:
an ultrasound probe;
a transceiver device configured to drive said ultrasound probe to scan a subject with an ultrasound beam;
an image generating device configured to generate an ultrasound image based on signals received by said transceiver device;
a display device configured to display the ultrasound image;
a first operation device and a second operation device configured to receive input instructions from an operator; and
a control device configured to receive the input instructions from said first operation device and said second operation device and to control said transceiver device, said image generating device, and said display device in accordance with the input instructions.

2. The ultrasound diagnostic apparatus according to claim 1, wherein the types of instructions that can be received by said control device from said second operation device are the same as the types of instructions that can be received by said control device from said first operation device.

3. The ultrasound diagnostic apparatus according to claim 1, wherein only some of the types of instructions that can be received by said control device from said first operation device are permitted as the types of instructions that can be received by said control device from said second operation device.

4. The ultrasound diagnostic apparatus according to claim 1, wherein the types of instructions that can be received by said control device from said second operation device are switchable between the same instructions that can be received by said control device from said first operation device and some of the types of instructions that can be received by said control device from said first operation device.

5. The ultrasound diagnostic apparatus according to claim 1, wherein the types of instructions that can be received by said control device from said second operation device are selectable.

6. The ultrasound diagnostic apparatus according to claim 2, wherein the types of instructions that can be received by said control device from said second operation device are selectable.

7. The ultrasound diagnostic apparatus according to claim 3, wherein the types of instructions that can be received by said control device from said second operation device are selectable.

8. The ultrasound diagnostic apparatus according to claim 4, wherein the types of instructions that can be received by said control device from said second operation device are selectable.

9. An ultrasound diagnostic apparatus comprising:
an ultrasound probe;
a transceiver device configured to drive said ultrasound probe to scan a subject with an ultrasound beam;
an image generating device configured to generate an ultrasound image based on the scan;
a first display device and a second display device configured to display the ultrasound image;
a first operation device and a second operation device configured to receive input instructions from an operator; and
a control device configured to receive the input instructions from said first operation device and said second operation device and to control said transceiver device, said image generating device, said first display device, and said second display device in accordance with the input instructions.

10. The ultrasound diagnostic apparatus according to claim 9, wherein said first display device cannot receive input instructions from said second operation device.

11. The ultrasound diagnostic apparatus according to claim 9, wherein said second display device cannot receive input instructions from said first operation device.

12. The ultrasound diagnostic apparatus according to claim 10, wherein said second display device cannot receive input instructions from said first operation device.

13. The ultrasound diagnostic apparatus according to claim 9, wherein the types of instructions that can be received by said control device from said second operation device do not include some of the types of instructions that can be received by said control device from said first operation device.

14. The ultrasound diagnostic apparatus according to claim 13, wherein the types of instructions that can be received by said control device from said second operation device are selectable.

15. The ultrasound diagnostic apparatus according to claim 14, wherein some of the types of instructions that can be received by said control device from said first operation device cannot be selected as the types of instructions that can be received by said control device from said second operation device.

16. The ultrasound diagnostic apparatus according to claim 9, wherein the types of instructions that can be received by said control device from said second operation device include all of the types of instructions that can be received by said control device from said first operation device.

17. The ultrasound diagnostic apparatus according to claim 9, wherein the types of instructions that can be inputted from said second operation device are switchable between all and not all of the types of instructions that can be inputted from said first operation device.

18. The ultrasound diagnostic apparatus according to claim 17, wherein the types of instructions that can be received by said control device from said second operation device are selectable.

19. The ultrasound diagnostic apparatus according to claim 18, wherein the types of instructions that can be received by said control device from said second operation device are selectable out of all of the types of instructions that can be received by said control device from said first operation device.

20. The ultrasound diagnostic apparatus according to claim 18, wherein, when the types of instructions that can be received by said control device from said second operation device are selected in a state in which the types of instructions that can be received by said control device from said second operation device have been so switched as not to include some of the types of instructions that can be received by said control device from said first operation device, some of the types of instructions that can be received by said control device from said first operation device cannot be selected.
